(19) United States
(12) Patent Application Publication
(10) Pub. No.: US 2018/0114922 A1
(43) Pub. Date: Apr. 26, 2018

(54) COMPOUND AND PREPARATION METHOD THEREOF, AND ORGANIC ELECTROLUMINESCENT DEVICE

(71) Applicants: BOE TECHNOLOGY GROUP CO., LTD., Beijing (CN); HEFEI XINSHENG OPTOELECTRONICS TECHNOLOGY CO., LTD., Hefei, Anhui (CN)

(72) Inventors: Jian CHEN, Beijing (CN); Sangman YUK, Beijing (CN); Bin LI, Beijing (CN); Tao ZHU, Beijing (CN); Huifang YUAN, Beijing (CN); Wenhao TANG, Beijing (CN); Qun FANG, Beijing (CN); Haibin YIN, Beijing (CN); Anxin DONG, Beijing (CN)

(73) Assignees: Boe Technology Group Co. Ltd., Beijing (CN); Hefei Xinsheng Optoelectronics Technology Co., Ltd, Anhui (CN)

(21) Appl. No.: 15/514,201

(22) PCT Filed: Jul. 13, 2016
(86) PCT No.: PCT/CN2016/089861

§ 371 Date: Mar. 24, 2017

(30) Foreign Application Priority Data
Jan. 4, 2016 (CN) 201610006395.5

(51) Int. Cl. H01L 51/00; C07D 409/04; C09K 11/02

(52) U.S. Cl. CPC H01L 51/0072; C07D 409/04; H01L 51/5016; C09K 11/025; H01L 51/0074

(57) ABSTRACT

Disclosed are a compound and a method for preparing the same, and an organic electroluminescent device. The compound has a general formula as shown in Formula I: wherein R is a substituent containing an N, O or S atom. The application of the compound to a host material of a light-emitting layer for preparing an organic electroluminescent device can improve the luminescence efficiency.

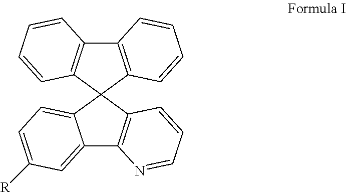

### TECHNICAL FIELD

The embodiments of the present invention relate to a compound and a preparation method thereof, and an organic electroluminescent device.

### BACKGROUND

An organic electroluminescent device is a self-luminous device, and includes a cathode, an anode and a luminescent layer provided between the cathode and the anode. When a voltage is applied to the organic electroluminescent device, the electrons injected from the cathode and the holes injected from the anode are combined at luminescent centers to form molecular excitons which release energy and luminesce when returning to a ground state. An organic electroluminescent device has a low voltage, a great brightness, a wide view angle, a fast response, good temperature adaptability and other characteristics, and thus is widely used in displays of electronic products such as TVs, cell phones and MP3 players.

Organic electroluminescent materials are typically classified as singlet fluorescent dyes and triplet phosphorescent dyes. However, both singlet fluorescent dyes and triplet phosphorescent dyes may have serious concentration quenching effects which will reduce the luminescence efficiency of a luminescent layer and result in a decrease in the luminescent performance of an organic luminescent device. Currently, the organic electroluminescent device generally has a host-guest structure, that is, a fluorescent dye or a phosphorescent dye is doped as a guest material into a host material at a certain concentration to avoid concentration quenching and triplet-triplet annihilation, thereby improving the luminescent performance of the device.

In 1999, Forrest, Thompson et al., doped a green phosphorescent material Ir(ppy)₃ at a concentration of 6 wt % into a host material of 4,4′-N,N′-dicarbazyl-biphenyl (CBP) to obtain a green organic electroluminescent device with a maximum external quantum efficiency of 10%. However, CBP has a triplet excited state energy of only 2.56 eV. If a blue phosphorescent material having a high triplet excited state energy is doped, energy would return to the host material, and thus the external quantum efficiency of the device would be reduced to be 5.7%. In order to further improve the luminescence efficiency of the blue phosphorescent device, a host material having high triplet energy needs to be used.

In 2003, Forrest developed N,N′-dicarbazyl-3,5-substituted benzene (mCP), which could contract the conjugated system of CBP and raise the triplet energy thereof to be 2.9 eV, thereby improving the external quantum efficiency of the device up to 7.8%. However, mCP has a low glass transition temperature and the stability of the material itself is not high. Furthermore, mCP exhibits imbalance of injected electrons and holes in the device, which results in an excess of holes in the device and thus reduces the luminescence efficiency of the organic electroluminescent device.

Therefore, it is desirable to provide a further compound as the host material of an organic electroluminescent device. The host material not only has great triplet energy, good stability, and a balance of electrons and holes, but also can improve the luminescence efficiency of the organic electroluminescent device.

### SUMMARY

The embodiments of the present invention relate to a compound and a preparation method thereof, and an organic electroluminescent device. The application of the compound to the host material of an organic electroluminescent device can solve the current technical problems of instable performance of host materials and imbalance of electrons and holes, and can improve the luminescence efficiency of the organic electroluminescent device.

At least one embodiment of the present invention provides a compound, having a general formula as shown in Formula I:

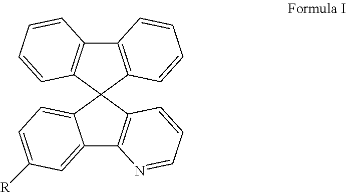

wherein R is a substituent containing an N, O or S atom.

For example, in compounds according to one embodiment of the present invention, R is one of the following groups:

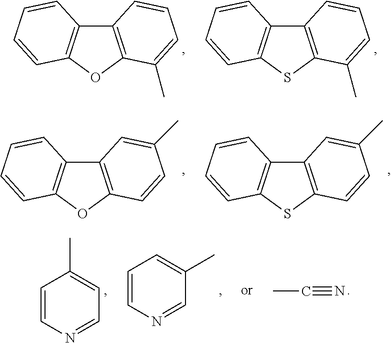

For example, in a compound according to one embodiment of the present invention, the compound is as shown in Formula I-1:

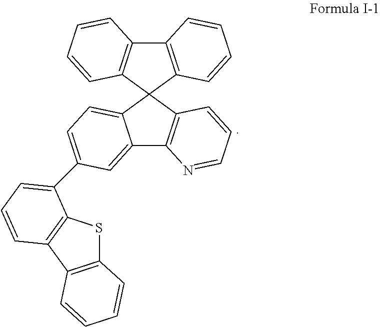 [SMILES: c1ccc2c(c1)-c1ccccc1C21c2ccc(-c3cccc4c3sc3ccccc34)cc2-c2ncccc21]

At least one embodiment of the present invention further provides a method of preparing the compound as shown in Formula I-1, comprising:

a step of coupling a compound as shown in Formula II with a compound as shown in Formula III so as to obtain the compound as shown in Formula I-1:

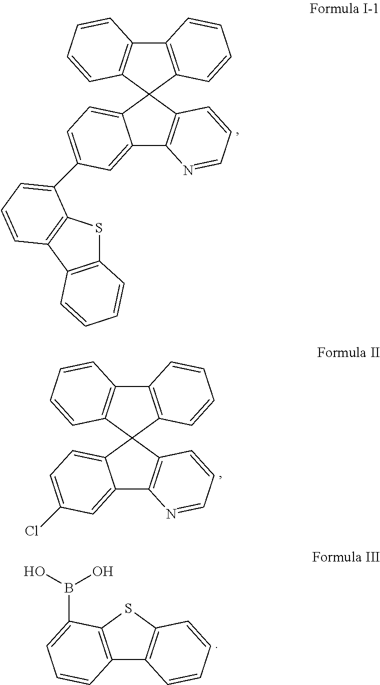

For example, in a method according to one embodiment of the present invention, a catalyst is used for catalyzation in the step of coupling.

For example, in a method according to one embodiment of the present invention, the catalyst comprises tetra(triphenyl phosphine) palladium.

For example, in a method according to one embodiment of the present invention, a molar ratio of the compound as shown in Formula II to the compound as shown in Formula III is from about 1:1.05 to about 1:1.2.

For example, in a method according to one embodiment of the present invention, the compound as shown in Formula II is obtained by reacting a compound as shown in Formula IV with a compound as shown in Formula V:

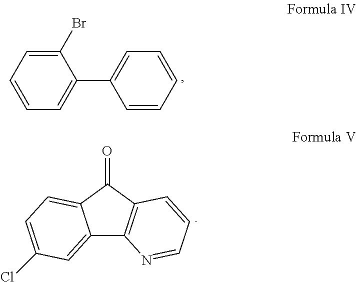

For example, in a method according to one embodiment of the present invention, a molar ratio of the compound as shown in Formula IV to the compound as shown in Formula V is from about 1:1.0 to about 1:2.0.

For example, in a method according to one embodiment of the present invention, the compound as shown in Formula V is obtained by reacting a compound as shown in Formula VI with a compound as shown in Formula VII:

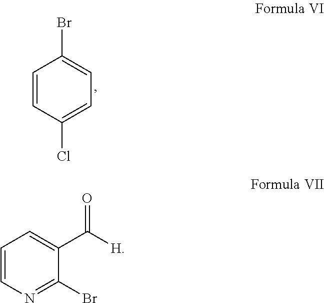

At least one embodiment of the present invention further provides an organic electroluminescent device, wherein a host material of the organic luminescent layer of the organic electroluminescent device comprises a compound as described above.

### BRIEF DESCRIPTION OF THE DRAWINGS

In order to clearly illustrate the technical solutions of the embodiments of the present invention, the drawings of the embodiments will be briefly described below. Apparently, the drawings described below relate to only some embodiments of the present invention, and thus are not limitative of the present invention.

FIG. 1 shows a structure of an organic light-emitting diode.

FIG. 2 shows the electroluminescence spectra of the organic light-emitting diodes in Example 4 and Comparative Example 4.

### DETAILED DESCRIPTION

To make clearer the objects, technical solutions and advantages of the embodiments of the present invention, a clear and detailed description of the technical solutions of the embodiments of the present invention will be made with reference to the accompanying drawings. Apparently, the described embodiments are just part of rather than all of the embodiments of the present invention. Based on the embodiments of the present invention described, all the other embodiments obtained by a person of ordinary skill in the art, without any creative labor, fall within the scope of protection of the present invention.

An embodiment of the present invention provides a compound, having a general formula as shown in Formula I:

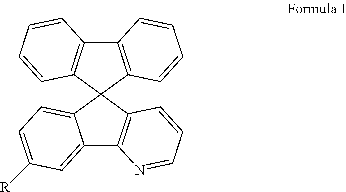

wherein R is a substituent containing an N, O or S atom.

For example, the compound contains a 4-aza spirocyclic group having a certain spatial structure. If the compound containing the group is applied to the host material of an organic luminescent layer in an organic electroluminescent device, the intermolecular aggregation effect can be reduced, that is, the concentration quenching can be reduced, thereby improving the luminescence efficiency of the organic light-emitting diode. The group contains an N atom having a strong electron withdrawing capability, which can improve the electron transport ability thereof, thereby ensuring the balance between the transported electrons and holes in the organic luminescent layer and improving the luminescence efficiency of the organic light-emitting diode. Furthermore, the compound containing the group has superior moldability and thermal stability, and the lifetime and luminescence efficiency of the organic electroluminescent device can be improved by applying this compound to the organic luminescent layer in the organic electroluminescent device such as an organic light-emitting diode.

For example, R is one of the following groups:

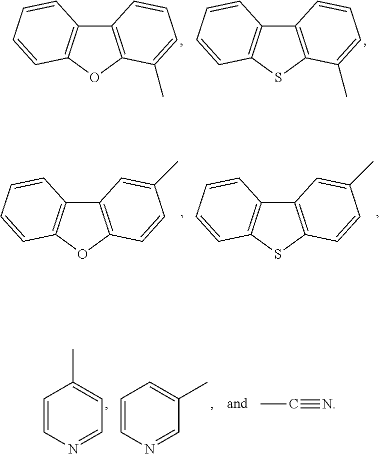

For example, R containing an N, O or S atom may be introduced into the compound. All of the N, O and S atoms have relatively strong electron withdrawing capability, which can improve the electron transport ability thereof. If the compound is applied to the organic luminescent layer, the balance between the transported electrons and holes can be ensured, thereby improving the luminescence efficiency of the organic light-emitting diode. For example, R may be further selected from the group consisting of carbazyl, diphenylamino, triphenylamino and diphenylphosphoryl.

For example, the compound is illustratively as shown in Formula I-1:

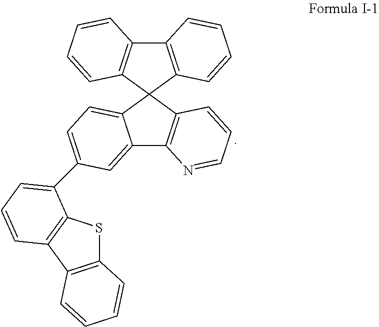 [SMILES: c1ccc2c(c1)-c1ccccc1C21c2ccc(-c3cccc4c3sc3ccccc34)cc2-c2ncccc21]

For example, 2-benzothiophene is introduced into the compound as a substituent. The substituent contains a sulfur atom which has a relatively strong electron withdrawing capability, which can improve the electron transport ability thereof. If the compound is applied to the organic electroluminescent device, the balance between the transported electrons and holes in the organic luminescent layer can be ensured, thereby improving the luminescence efficiency of the organic light-emitting diode.

An embodiment of the present invention further provides a method of preparing the compound as shown in Formula I-1, comprising:

a step of coupling a compound as shown in Formula II with a compound as shown in Formula III so as to obtain the compound as shown in Formula I-1:

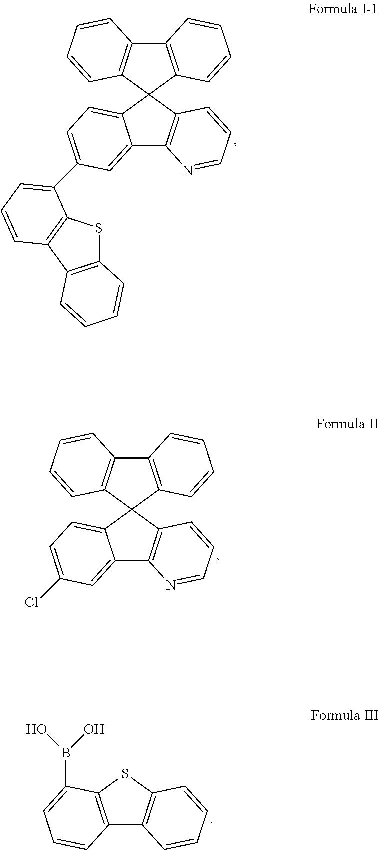 [SMILES: c1ccc2c(c1)-c1ccccc1C21c2ccc(-c3cccc4c3sc3ccccc34)cc2-c2ncccc21 ; Clc1ccc2c(c1)-c1ncccc1C21c2ccccc2-c2ccccc21 ; OB(O)c1cccc2c1sc1ccccc12]

For example, the compound as shown in Formula I-1 prepared by a method according to an embodiment of the present invention can have a yield of over 95%.

For example, a catalyst can be used for a catalytic reaction in the coupling step.

For example, the catalyst comprises tetra (triphenyl phosphine) palladium.

For example, when the compound as shown in Formula II and the compound as shown in Formula III are subjected to a coupling reaction, the molar ratio of the compound as shown in Formula II to the compound as shown in Formula III is from about 1:1.05 to about 1:1.2. In the coupling step, the compound as shown in Formula II and the compound as shown in Formula III within said molar ratio range would result in a great yield of the product, i.e., the compound as shown in Formula I-1.

For example, using tetra(triphenyl phosphine) palladium as a catalyst for a catalytic reaction in the coupling step would achieve a better effect.

For example, the coupling step is illustratively as follows: about 1.0 mole of the compound as shown in Formula II (6-chloro-4-aza-spirobifluorene) and from about 1.05 to about 1.2 moles of the compound as shown in Formula III (dibenzothiophene-4-boric acid) were added to a reactor, and tetra(triphenyl phosphine) palladium in a mass percent of about 0.5-5% of the compound as shown in Formula II was added as a catalyst; then, tetrahydrofuran was added as a solvent and a K₂CO₃ solution at a concentration of about 2 mol/L was added; The mixture was refluxed under a protective gas of argon for about 24 hours; after cooling, dichloromethane was used for extraction; the extracted organic layer was dried over anhydrous sodium sulfate, processed by rotary evaporator, and then purified by a silica gel column with dichloromethane/petroleum ether at a volume ratio of preferably about 1:5; the compound as shown in Formula I-1, 6-(4-dibenzothiophene)-4-aza-spirobifluorene, with a yield of greater than 95%, was obtained after being further processed by rotary evaporator.

For example, the compound as shown in Formula II may be obtained by reacting a compound as shown in Formula IV with a compound as shown in Formula V:

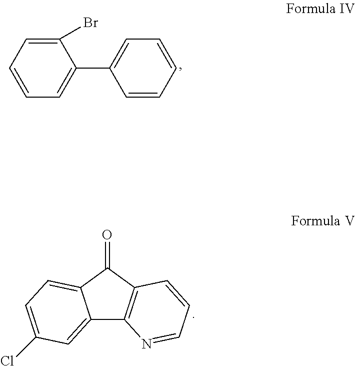 [SMILES: Brc1ccccc1-c1ccccc1 ; O=C1c2ccc(Cl)cc2-c2ncccc21]

For example, the compound as shown in Formula II obtained by the method can have a yield of over 80%.

For example, the molar ratio of the compound as shown in Formula IV to the compound as shown in Formula V is from about 1:1.0 to about 1:2.0. For example, when the compound as shown in Formula IV and the compound as shown in Formula V within said molar ratio range are reacted, the compound as shown in Formula II will have a greater yield.

For example, the step is illustratively as follows: about 1.0 mole of the compound as shown in Formula IV (2-bromo biphenyl) was added to a reactor; argon was used as the protective gas; anhydrous tetrahydrofuran was added as a solvent and then cooled to a temperature of from about −70° C. to about −78° C.; about 1.0-1.2 moles of a butyl lithium solution at a molar concentration of about 2 mol/L was slowly added dropwise; after reaction for about 5-8 hours, about 1.0-2.0 moles of the compound as shown in Formula V (4-aza-fluorenone) was added and further reacted for about 5-8 hours; anhydrous methanol was added for quenching, and then dichloromethane was added for extraction; the organic layer was dried over anhydrous sodium sulfate and then processed by rotary evaporator; to the obtained intermediate, a suitable amount of acetic acid and a suitable amount of hydrochloric acid were added directly, heated and refluxed for about 5-8 hours; the reaction was monitored by running a thin layer chromatography plate; after the reaction was determined to be over, dichloromethane was used for extraction; the organic layer was dried over anhydrous sodium sulfate and then processed by rotary evaporator; the crude product was purified with a column by using dichloromethane/petroleum ether at a volume ratio of preferably about 1:5; the compound as shown in Formula II, 6-chloro-4-aza-spirobifluorene, with a yield of greater than 80.5%, was obtained after being further processed by rotary evaporator.

For example, the compound as shown in Formula V was obtained by reacting a compound as shown in Formula VI with a compound as shown in Formula VII:

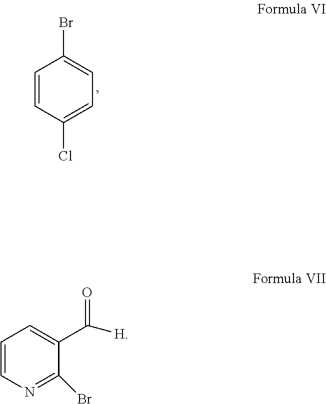

For example, the compound as shown in Formula V obtained by reacting the compound as shown in Formula VI with the compound as shown in Formula VII can have a yield of over 83.7%.

For example, an intermediate is formed by the compound as shown in Formula VI and the compound as shown in Formula VII in the presence of butyl lithium. The intermediate closes the ring in the presence of lead acetate to form the compound as shown in Formula V. The compound as shown in Formula V prepared under this condition has a higher yield.

For example, the step is illustratively as follows: the compound as shown in Formula VI (para-bromo-chlorobenzene) and the compound as shown in Formula VII were added to a reactor; anhydrous tetrahydrofuran was added as a solvent under a protective gas of argon, and then cooled to a temperature of about −70° C.˜−78° C.; about 1.0-2.0 moles of a butyl lithium solution at a molar concentration of about 2 mol/L was slowly added dropwise; after reaction for about 5-8 hours, anhydrous methanol was added for quenching, and then dichloromethane was added for extraction; the organic layer was dried over anhydrous sodium sulfate and then processed by rotary evaporator; to the obtained intermediate, a suitable amount of palladium acetate (for example, palladium acetate in a mass percent of about 20% of the compound as shown in Formula VI) and a suitable amount of anhydrous potassium acetate (for example, anhydrous potassium acetate in a mass percent of about 75% of the compound as shown in Formula VI) were added directly; a ring-closing reaction was performed in an acidic environment; the reaction was further carried out for about 10-12 hours, and monitored by running a thin layer chromatography plate; after the reaction was determined to be over, dichloromethane was added for extraction; the organic layer was dried over anhydrous sodium sulfate, processed by rotary evaporator and then purified with a column by using dichloromethane/petroleum ether (for example, at a volume ratio of about 1:5); 4-aza-fluorenone with a yield of greater than 83% was obtained after being further processed by rotary evaporator.

An embodiment of the present invention further provides an organic electroluminescent device. In the organic electroluminescent device, the host material of the organic luminescent layer is a compound having the general formula as shown in Formula I as described above. For example, in the organic electroluminescent device, the host material of the organic luminescent layer is the compound having the general formula as shown in Formula I-1 as described above.

For example, the organic electroluminescent device (such as an organic light-emitting diode) according to embodiments of the present invention can emit light normally, and the maximum luminance, the maximum current efficiency and the maximum power efficiency thereof can be significantly improved.

Examples 1-3 illustrate the preparation method of the compound having the general formula as shown in Formula I-1. The compounds used are shown as follows:

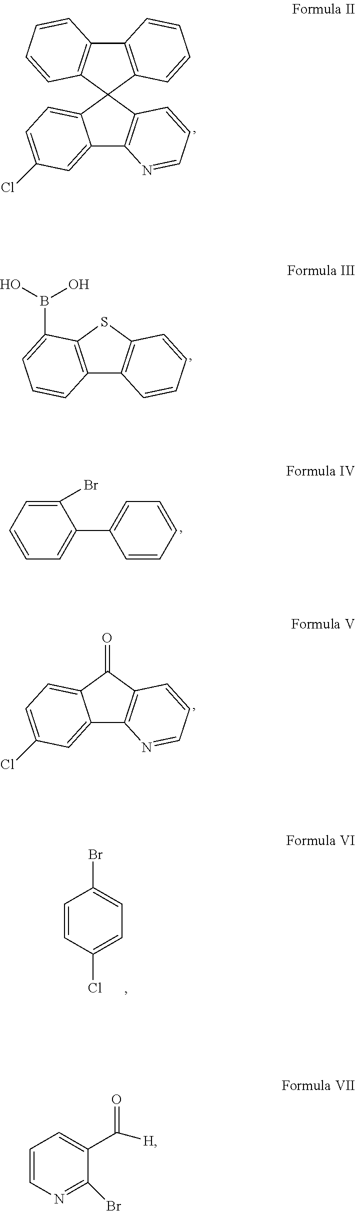

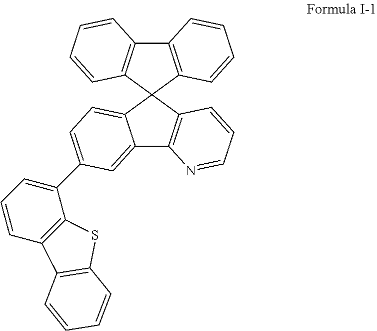

### Example 1

In this example, firstly, the compound as shown in Formula VI and the compound as shown in Formula VII were reacted to obtain the compound as shown in Formula V. The step was illustratively as follows: the compound as shown in Formula VI (para-bromo-chlorobenzene) and the compound as shown in Formula VII were added to a reactor; anhydrous tetrahydrofuran was added as a solvent under a protective gas of argon, and then cooled to about −70° C.; about 1.0 mole of a butyl lithium solution at a molar concentration of about 2 mol/L was slowly added dropwise; after reaction for about 5-8 hours, anhydrous methanol was added for quenching, and then dichloromethane was added for extraction; the organic layer was dried over anhydrous sodium sulfate and then processed by rotary evaporator; to the obtained intermediate, a suitable amount of palladium acetate and a suitable amount of anhydrous potassium acetate were added directly; the reaction was further carried out for about 10 hours, and monitored by running a thin layer chromatography plate; after the reaction was determined to be over, dichloromethane was added for extraction; the organic layer was dried over anhydrous sodium sulfate, processed by rotary evaporator, and then purified with a column by using dichloromethane/petroleum ether (at a volume ratio of about 1:5); 4-aza-fluorenone was obtained after being further processed by rotary evaporator.

Afterwards, the compound as shown in Formula IV and the compound as shown in Formula V were reacted to obtain the compound as shown in Formula II. The step was illustratively as follows: about 1.0 mole of the compound as shown in Formula IV (2-bromo biphenyl) was added to a reactor; argon was used as the protective gas; anhydrous tetrahydrofuran was added as a solvent and then cooled to about −70° C.; about 1.0 mole of a butyl lithium solution at a molar concentration of about 2 mol/L was slowly added dropwise; after reaction for about 5 hours, about 1.0 mole of the compound as shown in Formula V (4-aza-fluorenone) was added and further reacted for about 5 hours; anhydrous methanol was added for quenching, and then dichloromethane was added for extraction; the organic layer was dried over anhydrous sodium sulfate and then processed by rotary evaporator; to the obtained intermediate a suitable amount of acetic acid and a suitable amount of hydrochloric acid were added directly, heated and refluxed for about 5 hours in an acidic environment; the reaction was monitored by running a thin layer chromatography plate; after the reaction was determined to be over, dichloromethane was used for extraction; the organic layer was dried over anhydrous sodium sulfate and then processed by rotary evaporator; the crude product was purified with a column by using dichloromethane/petroleum ether at a volume ratio of about 1:5; the compound as shown in Formula II, 6-chloro-4-aza-spirobifluorene, was obtained after being further processed by rotary evaporator.

Finally, the compound as shown in Formula II and the compound as shown in Formula III were subjected to a coupling reaction to obtain the compound as shown in Formula I-1. The step was illustratively as follows: about 1.0 mole of the compound as shown in Formula II (6-chloro-4-aza-spirobifluorene) and about 1.05 moles of the compound as shown in Formula III (dibenzothiophene-4-boric acid) were added to a reactor, and tetra(triphenyl phosphine) palladium in a mass percent of about 0.5% of the compound as shown in Formula II was added as a catalyst; then, tetrahydrofuran was added as a solvent and a K₂CO₃ solution at a molar concentration of about 2 mol/L was added and refluxed for about 24 hours under a protective gas of argon; after cooling, dichloromethane was used for extraction; the extracted organic layer was dried over anhydrous sodium sulfate, processed by rotary evaporator and then purified with a silica gel column by using dichloromethane/petroleum ether at a volume ratio of about 1:5; the compound as shown in Formula I-1, 6-(4-dibenzothiophene)-4-aza-spirobifluorene, was obtained after being further processed by rotary evaporator.

### Example 2

In this example, firstly, the compound as shown in Formula VI and the compound as shown in Formula VII were reacted to obtain the compound as shown in Formula V. The step was illustratively as follows: the compound as shown in Formula VI (para-bromo-chlorobenzene) and the compound as shown in Formula VII were added into a reactor; anhydrous tetrahydrofuran was added as a solvent under a protective gas of argon, and then cooled to about −78° C.; about 2.0 mole of a butyl lithium solution at a molar concentration of about 2 mol/L was slowly added dropwise; after reaction for about 8 hours, anhydrous methanol was added for quenching, and then dichloromethane was added for extraction; the organic layer was dried over anhydrous sodium sulfate and then processed by rotary evaporator; to the obtained intermediate, a suitable amount of palladium acetate and a suitable amount of anhydrous potassium acetate were added directly; the reaction was further carried out for about 12 hours, and monitored by running a thin layer chromatography plate; after the reaction was determined to be over, dichloromethane was added for extraction; the organic layer was dried over anhydrous sodium sulfate, processed by rotary evaporator, and then purified with a column by using dichloromethane/petroleum ether (at a volume ratio of about 1:5); 4-aza-fluorenone was obtained after being further processed by rotary evaporator.

Afterwards, the compound as shown in Formula IV and the compound as shown in Formula V were reacted to obtain the compound as shown in Formula II. The step was illustratively as follows: 1.0 mole of the compound as shown in Formula IV (2-bromo biphenyl) was added to a reactor; argon was used as the protective gas; anhydrous tetrahydrofuran was added as a solvent and then cooled to about −78° C.; about 1.2 mole of a butyl lithium solution at a molar concentration of about 2 mol/L was slowly added dropwise; after reaction for about 8 hours, about 2.0 moles of the compound as shown in Formula V (4-aza-fluorenone) was added and further reacted for about 8 hours; anhydrous methanol was added for quenching, and then dichloromethane was added for extraction; the organic layer was dried over anhydrous sodium sulfate and then processed by rotary evaporator; to the obtained intermediate, a suitable amount of acetic acid and a suitable amount of hydrochloric acid were added directly, heated and refluxed for about 8 hours in an acidic environment; the reaction was monitored by running a thin layer chromatography plate; after the reaction was determined to be over, dichloromethane was used for extraction; the organic layer was dried over anhydrous sodium sulfate and then processed by rotary evaporator; the crude product was purified with a column by using dichloromethane/petroleum ether at a volume ratio of about 1:5; the compound as shown in Formula II, 6-chloro-4-aza-spirobifluorene, was obtained after being further processed by rotary evaporator.

Finally, the compound as shown in Formula II and the compound as shown in Formula III were subjected to a coupling reaction to obtain the compound as shown in Formula I-1. The step was illustratively as follows: about 1.0 mole of the compound as shown in Formula II (6-chloro-4-aza-spirobifluorene) and about 1.2 moles of the compound as shown in Formula III (dibenzothiophene-4-boric acid) were added to a reactor, and tetra(triphenyl phosphine) palladium in a mass percent of about 5% of the compound as shown in Formula II was added as a catalyst; then, tetrahydrofuran was added as a solvent and a K₂CO₃ solution at a molar concentration of about 2 mol/L was added and refluxed for about 24 hours under a protective gas of argon; after cooling, dichloromethane was used for extraction; the extracted organic layer was dried over anhydrous sodium sulfate, processed by rotary evaporator, and then purified with a silica gel column by using dichloromethane/petroleum ether at a volume ratio of about 1:5; the compound as shown in Formula I-1, 6-(4-dibenzothiophene)-4-aza-spirobifluorene, was obtained after being further processed by rotary evaporator.

### Example 3

In this example, firstly, the compound as shown in Formula VI and the compound as shown in Formula VII were reacted to obtain the compound as shown in Formula V. The step was illustratively as follows: the compound as shown in Formula VI (para-bromo-chlorobenzene) and the compound as shown in Formula VII were added to a reactor; argon was used as the protective gas; anhydrous tetrahydrofuran was added as a solvent, and then cooled to about −75° C.; about 1.5 moles of a butyl lithium solution at a molar concentration of about 2 mol/L was slowly added dropwise; after reaction for about 6 hours, anhydrous methanol was added for quenching, and then dichloromethane was added for extraction; the organic layer was dried over anhydrous sodium sulfate and then processed by rotary evaporator; to the obtained intermediate a suitable amount of palladium acetate and a suitable amount of anhydrous potassium acetate were added directly; the reaction was further carried out for about 11 hours, and monitored by running a thin layer chromatography plate; after the reaction was determined to be over, dichloromethane was added for extraction; the organic layer was dried over anhydrous sodium sulfate, processed by rotary evaporator, and then purified with a column by using dichloromethane/petroleum ether (at a volume ratio of about 1:5); 4-aza-fluorenone was obtained after being further processed by rotary evaporator.

Afterwards, the compound as shown in Formula IV and the compound as shown in Formula V were reacted to obtain the compound as shown in Formula II. The step was illustratively as follows: about 1.0 mole of the compound as shown in Formula IV (2-bromo biphenyl) was added to a reactor; argon was used as the protective gas; anhydrous tetrahydrofuran was added as a solvent and then cooled to about −74° C.; about 1.1 mole of a butyl lithium solution at a molar concentration of about 2 mol/L was slowly added dropwise; after reaction for about 6 hours, about 1.5 moles of the compound as shown in Formula V (4-aza-fluorenone) were added and further reacted for about 6 hours; anhydrous methanol was added for quenching, and then dichloromethane was added for extraction; the organic layer was dried over anhydrous sodium sulfate and then processed by rotary evaporator; to the obtained intermediate a suitable amount of acetic acid and a suitable amount of hydrochloric acid were added directly, heated and refluxed for about 7 hours in an acidic environment; the reaction was monitored by running a thin layer chromatography plate; after the reaction was determined to be over, dichloromethane was used for extraction; the organic layer was dried over anhydrous sodium sulfate and then processed by rotary evaporator; the crude product was purified with a column by using dichloromethane/petroleum ether at a volume ratio of about 1:5; the compound as shown in Formula II, 6-chloro-4-aza-spirobifluorene, was obtained after being further processed by rotary evaporator.

Finally, the compound as shown in Formula II and the compound as shown in Formula III were subjected to a coupling reaction to obtain the compound as shown in Formula I-1. The step was illustratively as follows: about 1.0 mole of the compound as shown in Formula II (6-chloro-4-aza-spirobifluorene) and about 1.1 moles of the compound as shown in Formula III (dibenzothiophene-4-boric acid) were added to a reactor, tetra(triphenyl phosphine) palladium in a mass percent of about 3% of the compound as shown in Formula II was added as a catalyst, tetrahydrofuran was added as a solvent and a K₂CO₃ solution at a molar concentration of about 2 mol/L was added, and then refluxed for about 24 hours under a protective gas of argon; after cooling, dichloromethane was used for extraction; the extracted organic layer was dried over anhydrous sodium sulfate, processed by rotary evaporator and then purified with a silica gel column by using dichloromethane/petroleum ether at a volume ratio of about 1:5; the compound as shown in Formula I-1, 6-(4-dibenzothiophene)-4-aza-spirobifluorene, was obtained after being further processed by rotary evaporator.

The compound as shown in Formula I-1 could be prepared by using any method in Examples 1-3 of the present invention, and the yield of the compound as shown in Formula I-1 was greater than 95%.

The compound obtained in Example 3 was subjected to nuclear magnetic spectrogram analysis, resulting in the following data: ¹HNMR (400 MHz, CDCl₃): 8.48-8.41 (m, 3H), 8.20 (d, J=8.5, 1H), 8.09 (s, 1H), 7.98 (s, 1H), 7.87 (d, J=8.5, 1H), 8.50-8.58 (m, 5H), 7.28-7.38 (m, 7H), 6.67 (s, 1H). Thus, it can be determined that the compound obtained was the compound as shown in Formula I-1.

The compounds HAT-CN, TAPC, Firpic, TmpypB, mCP, Liq and TPBi, involved in the following description, were respectively represented as follows:

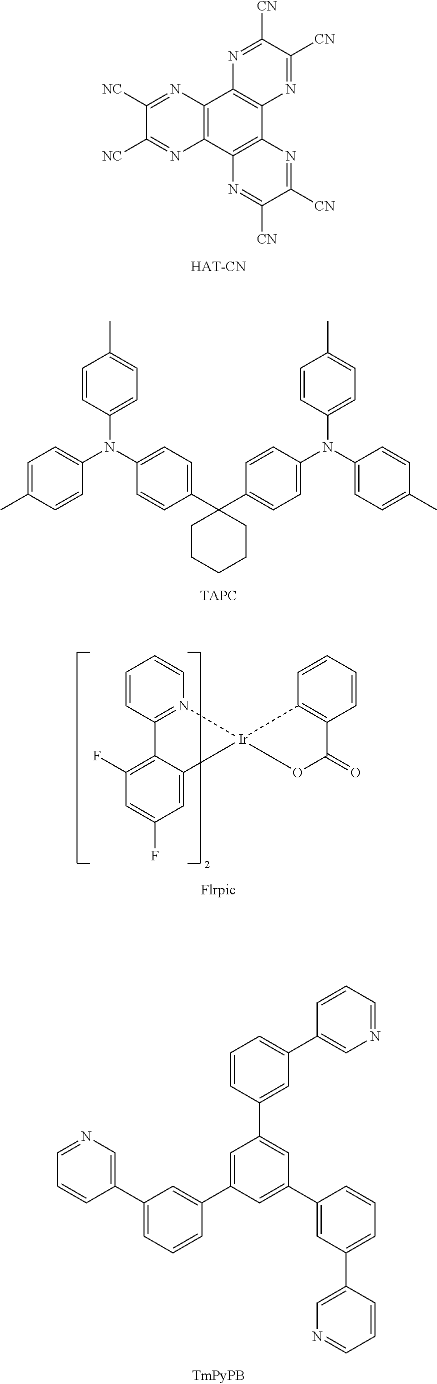

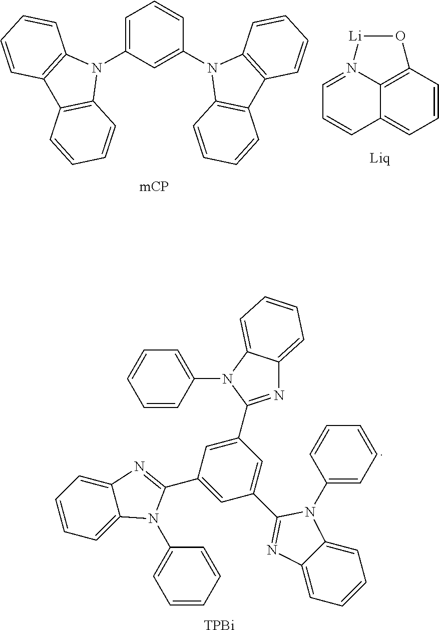

Organic electroluminescent devices prepared by using the compound of the present invention may comprise an organic light-emitting diode.

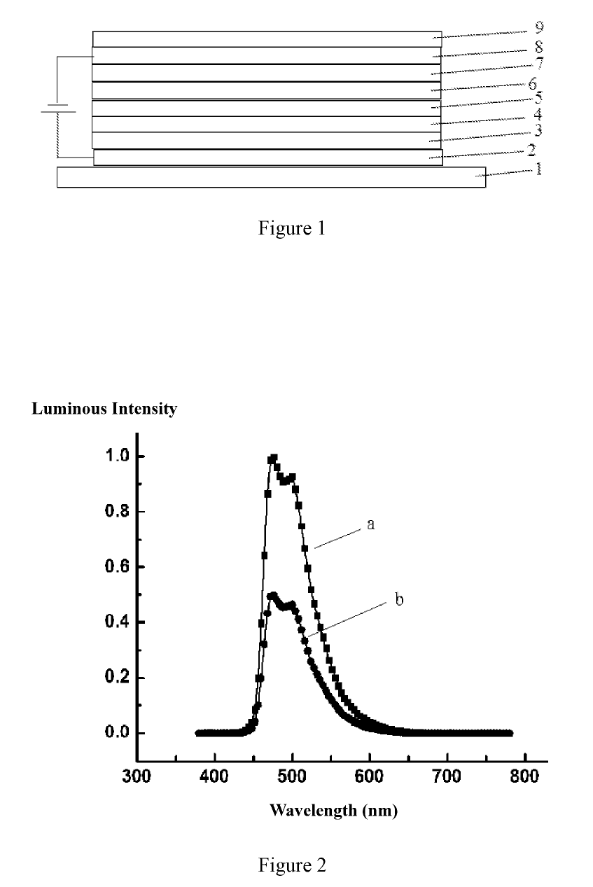

As shown in FIG. 1, the structure of the organic light-emitting diode includes: a substrate 1, an anode layer 2, a hole injection layer 3, a hole transport layer 4, an organic luminescent layer 5, an electron transport layer 6, an electron injection layer 7, a cathode layer 8 and an encapsulating film 9. An ITO transparent conductive glass substrate was used as the substrate 1; molybdenum trioxide (MoO₃) or 2,3,6,7,10,11-hexacyano-1,4,5,8,9,12-hexaaza-triphenylene (HAT-CN) was used as the hole injection layer; nitrophenol butylate (NPB), CBP or TAPC was used as the hole transport layer; TPBi, 4,7-diphenyl-1,10-phenanthroline (Bphen) or 1,3,5-tri[(3-pyridyl)-3-phenyl]benzene (TmPyPB) was used as the electron transport layer; the organic luminescent layer of the organic light-emitting diode could be single-layered or multi-layered, where each luminescent layer could have a single-doped structure or a multiple-doped structure. The luminescent color was not limited, for example red, yellow, blue, green or white. Phosphorescence dyes included FIrpic, tri(2-phenylpyridine) iridium (Ir(ppy)₃), acetopyruvic acid di(2-phenylpyridine) iridium (Ir(ppy)2(acac)), tri(1-phenyl-isoquinoline) iridium (Ill) (Ir(piq)) or (acetylacetone) bis(2-methyl-dibenzo[F,H]quinoxaline) iridium (Ir(MDQ)₂(acac)), etc. The structure of a metal or metal mixture (such as Mg:Al, Li:Al, etc.) could be used for the cathode, or a conventional cathode structure of an electron injection layer/metal layer (such as LiF/Al, Liq/Al) could be adopted, wherein the electron injection layer might be an element, compound or mixture of an alkali metal, an alkaline earth metal, or a transition metal.

### Example 4

In this example, an organic light-emitting diode was prepared by using the above-mentioned compound as the host material of the luminescent layer. In Comparative Example 4, mCP was adopted as the host material to prepare an organic light-emitting diode, and all the other conditions were completely the same as those in Example 4. Then, the properties of the organic light-emitting diodes obtained were detected.

For example, the compound as shown in Formula I-1 in the embodiments of the present invention was used as the host material of the organic luminescent layer in the organic light-emitting diode. FIrpic was a blue phosphorescence dye. The structure of the organic light-emitting diode was as shown in FIG. 1, which comprised a substrate 1, an anode layer 2, a hole injection layer 3, a hole transport layer 4, an organic luminescent layer 5, an electron transport layer 6, an electron injection layer 7, a cathode layer 8 and an encapsulating film 9, wherein an ITO transparent conductive glass substrate was used as the substrate 1; HAT-CN with a thickness of 10 nm was used as the hole injection layer 3; TAPC with a thickness of 45 nm was used as the hole transport layer 4; the host material of the organic luminescent layer was the compound as shown in Formula I-1; the guest material was the FIrpic blue phosphorescence dye with a doping concentration of 8%; TmPyPB with a thickness of 40 nm was used as the electron transport layer 6; Liq with a thickness of 2 nm was used as the electron injection layer 7; and Al with a thickness of 120 nm was used as the cathode layer 8.

The preparation process of the organic light-emitting diode was as follows: the ITO transparent conductive glass substrate 1 was placed into a detergent for ultrasonic treatment, rinsed with deionized water, then washed three times repeatedly with deionized water, acetone and ethanol, baked in a clean environment to remove water completely, and finally treated with an ultraviolet lamp and ozone. The treated ITO transparent conductive glass substrate was placed into a vacuum cavity which was evacuated to about 3.0×10⁻⁴ to 4.0×10⁻⁴ Pa. HAT-CN was vacuum evaporated onto the ITO transparent conductive glass substrate as a hole injection layer at an evaporation rate of 0.25 Å/s, where the coated film had a thickness of 10 nm. TAPC was vacuum evaporated onto the hole injection layer as a hole transport layer at an evaporation rate of 2 Å/s, where the coated film had a thickness of 45 nm. Then, by using a double source evaporation process method, the organic luminescent layer was prepared by using Compound I-1 in the embodiments of the present invention as the host material and using a FIrpic blue phosphorescence dye as the guest material, where the evaporation rate was controlled to 2 Å/s, the thickness of the coated film was controlled to 20 nm, and the doping concentration of FIrpic was controlled to 8%. TmPyPB was vacuum evaporated onto the organic luminescent layer as the electron transport layer at an evaporation rate of 2 Å/s, where the coated film had a thickness of 40 nm. Liq and Al were vacuum evaporated as the electron injection layer and the cathode respectively on the electron transport layer.

### Comparative Example 4

In this comparative example, the compound mCP was used as the host material of the organic light-emitting diode. The structure and preparation process of the device were otherwise the same as those in Example 4.

The current-brightness-voltage characteristics of the organic light-emitting diode were obtained by a Keithley source measuring system (Keithley 2400 Sourcemeter, Keithley 2000 Currentmeter) provided with a corrected silicon photodiode; the electroluminescence spectrum was determined by a PR655 spectrometer available from the Photo Research Company. All the measurement was performed under the conditions of normal pressure and temperature.

According to the electroluminescence spectra of the organic light-emitting diodes of Example 4 (Curve a) and Comparative Example 4 (Curve b) as shown in FIG. 2, it can be seen that both the organic light-emitting diodes prepared in Example 4 and Comparative Example 4 could emit light normally, wherein the luminescence efficiency of the organic light-emitting diode in Example 4 was greater than that in Comparative Example 4.

The maximum brightness, the maximum current efficiency and the maximum power efficiency in Example 4 and Comparative Example 4 were tested. The results were as shown in Table 1:

TABLE 1

| Organic | | | |
|---|---|---|---|
| light-emitting | Maximum | Maximum current | Maximum power |
| diode | brightness, cd/m² | efficiency, cd/A | efficiency, lm/W |
| Example 4 | 11220 | 44.0 | 40.1 |
| Comparative | 5494 | 20.0 | 12.3 |
| Example 4 | | | |

It can be seen from the data in Table 1 that the maximum brightness, the maximum current efficiency and the maximum power efficiency in Example 4 were greater than those in Comparative Example 4. As such, it is concluded that using the compounds in the embodiments of the present invention as the host material of the organic luminescent layer of the organic light-emitting diode can efficiently improve the luminescence efficiency of the organic light-emitting diode, and the compounds in the embodiments of the present invention are suitable as host materials of the organic luminescent layer of the organic light-emitting diode.

The beneficial effects of the embodiments of the present invention are as follows:

The compounds in the embodiments of the present invention can be used as host materials of organic luminescent layers in organic electroluminescent devices. The compounds in the embodiments of the present invention, as host materials, have greater triplet energy, which can improve the external quantum efficiency of the organic electroluminescent device. As host materials, they have a high glass transition temperature and stable performance, and can balance the injection of electrons and holes and improve the luminescence efficiency of the organic electroluminescent device.

Apparently, the present invention may be modified or varied in many modes by a person of ordinary skill in the art without departing from the spirit and scope of the present invention. If such modifications or variations are within the scope of the claims of the present invention or their equivalent techniques, then the present invention also encompasses these modifications or variations.

The above are merely exemplary embodiments of the present invention, and are not intended to limit the scope of protection of the present invention, which is yet determined by the appended claims.

The present application claims the priority of the Chinese Patent Application No. 201610006395.5 submitted on Jan. 4, 2016, and the content disclosed in the above Chinese patent application is incorporated herein by reference as part of the present application.

1. A compound, having a general formula as shown in Formula I: 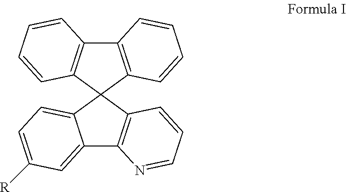
wherein R is a substituent containing an N, O or S atom.

2. The compound according to claim 1, wherein R is one of the following groups:

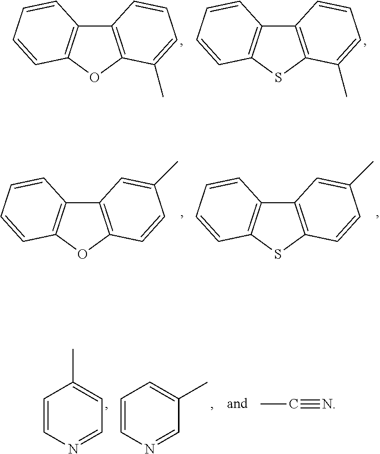

3. The compound according to claim 2, wherein the compound is as shown in Formula I-1:

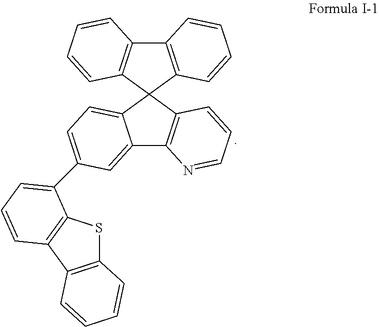 [SMILES: c1ccc2c(c1)-c1ccccc1C21c2ccc(-c3cccc4c3sc3ccccc34)cc2-c2ncccc21]

4. A method of preparing a compound as shown in Formula I-1, comprising:
a step of coupling a compound as shown in Formula II with a compound as shown in Formula III so as to obtain the compound as shown in Formula I-1:

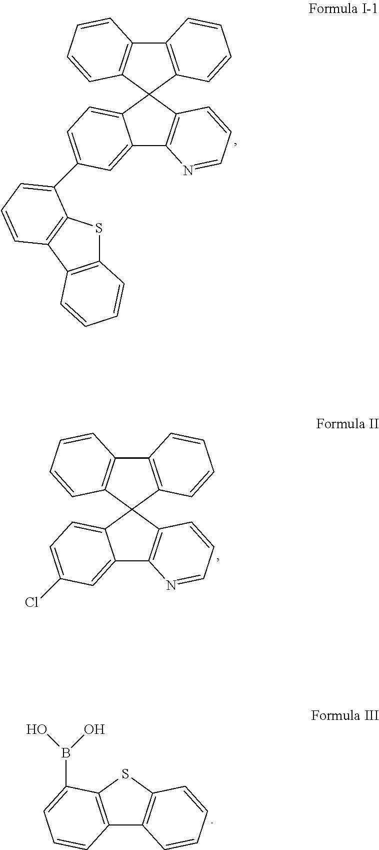 [SMILES: c1ccc2c(c1)-c1ccccc1C21c2ccc(-c3cccc4c3sc3ccccc34)cc2-c2ncccc21 ; Clc1ccc2c(c1)-c1ncccc1C21c2ccccc2-c2ccccc21 ; OB(O)c1cccc2c1sc1ccccc12]

5. The method according to claim 4, wherein a catalyst is used for catalyzation in the step of coupling.

6. The method according to claim 5, wherein the catalyst comprises tetra(triphenyl phosphine) palladium.

7. The method according to claim 4, wherein a molar ratio of the compound as shown in Formula II to the compound as shown in Formula III is from about 1:1.05 to about 1:1.2.

8. The method according to claim 4, wherein the compound as shown in Formula II is obtained by reacting a compound as shown in Formula IV with a compound as shown in Formula V:

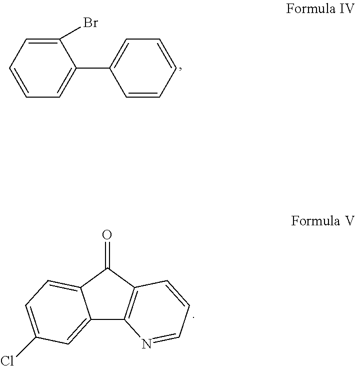 [SMILES: Brc1ccccc1-c1ccccc1 ; O=C1c2ccc(Cl)cc2-c2ncccc21]

9. The method according to claim 8, wherein a molar ratio of the compound as shown in Formula IV to the compound as shown in Formula V is from about 1:1.0 to about 1:2.0.

10. The method according to claim 8, wherein the compound as shown in Formula V is obtained by reacting a compound as shown in Formula VI with a compound as shown in Formula VII:

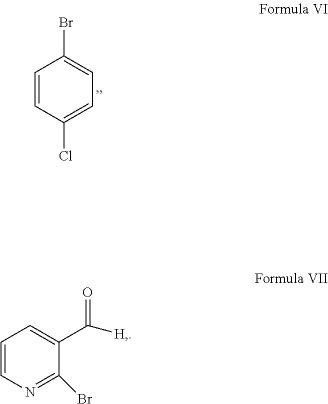

11. An organic electroluminescent device, comprising a cathode and an anode which are arranged oppositely, and an organic luminescent layer provided between the cathode and the anode, wherein a host material of the organic luminescent layer is the compound according to claim 1.

12. The organic electroluminescent device according to claim 11, wherein the host material of the organic luminescent layer is a compound having a general formula as shown in Formula I: 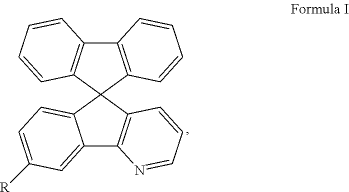
wherein R is one of the following groups:

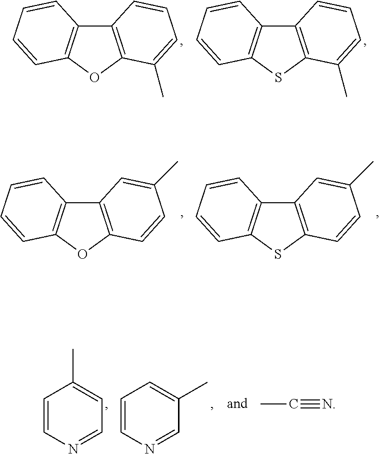

13. The organic electroluminescent device according to claim 12, wherein the compound is as shown in Formula I-1: 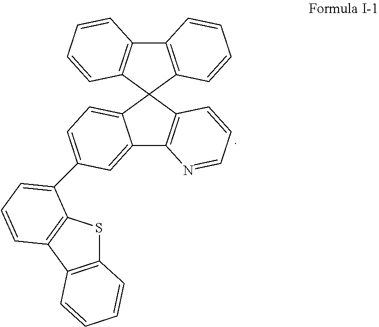 [SMILES: c1ccc2c(c1)-c1ccccc1C21c2ccc(-c3cccc4c3sc3ccccc34)cc2-c2ncccc21]
